(19) United States
(12) Patent Application Publication
(10) Pub. No.: US 2007/0190542 A1
(43) Pub. Date: Aug. 16, 2007

(54) HYBRIDIZATION ASSISTED NANOPORE SEQUENCING

(76) Inventors: Xinsheng Sean Ling, East Greenwich, RI (US); Barrett Bready, Providence, RI (US)

Correspondence Address: BARLOW, JOSEPHS & HOLMES, LTD., 101 DYER STREET, 5TH FLOOR, PROVIDENCE, RI, 02903, US

(21) Appl. No.: 11/538,189

(22) Filed: Oct. 3, 2006

Related U.S. Application Data
(60) Provisional application No. 60/723,284, filed on Oct. 3, 2005.
(60) Provisional application No. 60/723,207, filed on Oct. 28, 2005.

(51) Int. Cl. C12Q 1/68

(52) U.S. Cl. 435/6; 977/924

(57) ABSTRACT

A method of employing a nanopore structure in a manner that allows the detection of the positions (relative and/or absolute) of nucleic acid probes that are hybridized onto a single-stranded nucleic acid molecule. In accordance with the method the strand of interest is hybridized with a probe having a known sequence. The strand and hybridized probes are translocated through a nanopore. The fluctuations in current measured across the nanopore will vary as a function of time corresponding to the passing of a probe attachment point along the strand. These fluctuations in current are then used to determine the attachment positions of the probes along the strand of interest. This probe position data is then fed into a computer algorithm that returns the sequence of the strand of interest.

### CROSS-REFERENCE TO RELATED APPLICATIONS

This application is related to and claims priority from earlier filed U.S. Provisional Patent Application No. 60/723,284, filed Oct. 3, 2005 and earlier filed U.S. Provisional Application No. 60/723,207, filed Oct. 28, 2005, the contents of which are entirely incorporated herein by reference.

### STATEMENT REGARDING FEDERALLY SPONSORED RESEARCH OR DEVELOPMENT

The U.S. Government has a paid-up license in this invention and the right in limited circumstances to require the patent owner to license to others on reasonable terms as provided for by the terms of NSF-NIRT Grant No. 0403891 awarded by the National Science Foundation (NSF) Nanoscale Interdisciplinary Research Team (NIRT).

### BACKGROUND OF THE INVENTION

The present invention relates generally to a method of detecting, sequencing and characterizing biomolecules such as Deoxyribonucleic acid (DNA), Ribonucleic acid (RNA) and/or proteins. More specifically, the present invention is directed to a method of drawing a biomolecule through a membrane in a manner that allows the composition of the molecule to be identified and sequenced.

Currently, there is a great deal of interest in developing the ability to identify with specificity the composition and sequence of various biomolecules because such molecules are the fundamental building blocks of life. The ability to sequence and map the structures of these molecules leads to a greater understanding of the basic principles of life as well as the opportunity to develop an understanding of scores of genetically triggered diseases and conditions that until now have defied understanding and/or treatment. The difficulty is that in using prior art sequencing technology to sequence a single persons DNA, such as was done in the Human Genome Project, over $3 Billion dollars were expended. While this was a monumental and historic undertaking, it is estimated that each person's DNA varies from one another by approximately 1 base in 1000. It is this variation in bases that will allow the scientific community to identify genetic trends that are related to various predispositions and/or conditions. Therefore in order to obtain meaningful information the genetic code of millions of people must be sequenced thereby identifying the relevant regions where they differ.

There are numerous methods available in the prior art for use in connection with the sequencing of biomolecules of interest. The difficulty with these prior art methods however is that many of them are time consuming and expensive and as a result are not fully implemented, thereby limiting their potential. In the context of the present application, those biomolecule sequencing methods that are of particular interest are those that employ nanopore/micropore devices to accomplish the biomolecule sequencing. In this regard, nanopores are holes having diameters in the range of between approximately 200 nm to 1 nm that are formed in a membrane or solid media. Many applications have been contemplated in connection with the use of nanopores for the rapid detection and characterization of biological agents and DNA sequencing. In addition, larger micropores are already widely used as a mechanism for separating cells.

Two prior art DNA sequencing methods have been proposed using nanopores. U.S. Pat. No. 5,795,782, issued to Church et al., for example, discloses a method of reading a DNA sequence by detecting the ionic current variations as a single-stranded DNA molecule moves through a nanopore under a bias voltage. The difficulty with these methods is that the sequencing operation is performed on single-stranded DNA on a base-by-base operation. In this regard the inherent limitation is that it is nearly impossible to detect a significant enough change in signal as each base passes through the nanopore because there simply is not enough of a signal differential between each of the discrete base pairs. Further, using present day techniques it is nearly impossible to form a nanopore in a membrane thin enough to measure one base at a time.

Another method for DNA sequencing using nanopores was discussed in U.S. Pat. No. 6,537,755, issued to Drmanac. Drmanac proposes using nanopores to detect the DNA hybridization probes (oligonucleotides) on a DNA molecule and recover the DNA sequence information using the method of Sequencing-By-Hybridization (SBH). The classical SBH procedure attaches a large set of single-stranded fragments or probes to a substrate, forming a sequencing chip. A solution of labeled single-stranded target DNA fragments is exposed to the chip. These fragments hybridize with complementary fragments on the chip, and the hybridized fragments can be identified using a nuclear detector or a fluorescent/phosphorescent dye, depending on the selected label. Each hybridization or the lack thereof determines whether the string represented by the fragment is or is not a substring of the target. The target DNA can now be sequenced based on the constraints of which strings are and are not substrings of the target. Sequencing by hybridization is a useful technique for general sequencing, and for rapidly sequencing variants of previously sequenced molecules. Furthermore, hybridization can provide an inexpensive procedure to confirm sequences derived using other methods.

The most widely used sequencing chip design, the classical sequencing chip contains 65,536 octamers. The classical chip suffices to reconstruct 200 nucleotide-long sequences in only 94 of 100 cases, even in error-free experiments. Unfortunately, the length of unambiguously reconstructible sequences grows slower than the area of the chip. Thus, such exponential growth of the area inherently limits the length of the longest reconstructible sequence by classical SBH, and the chip area required by any single, fixed sequencing array on moderate length sequences will overwhelm the economies of scale and parallelism implicit in performing thousands of hybridization experiments simultaneously when using classical SBH methods. Other variants of SBH and positional SBH have been proposed to increase the resolving power of classical SBH, but these methods still require large arrays to sequence relatively few nucleotides.

The algorithmic aspect of sequencing by hybridization arises in the reconstruction of the test sequence from the hybridization data. The outcome of an experiment with a classical sequencing chip assigns to each of the strings a probability that it is a substring of the test sequence. In an experiment without error, these probabilities will all be 0 or 1, so each nucleotide fragment of the test sequence is unambiguously identified.

Although efficient algorithms do exist for finding the shortest string consistent with the results of a classical sequencing chip experiment, these algorithms have not proven useful in practice because previous SBH methods do not return sufficient information to sequence long fragments. One particular obstacle inherent in this method is the inability to accurately position repetitive sequences in DNA fragments. Furthermore, this method cannot determine the length of tandem short repeats, which are associated with several human genetic diseases. These limitations have prevented its use as a primary sequencing method

There is therefore a need for an improved method of sequencing organic biomolecules that can be accomplished at a higher throughput and with a higher degree of accuracy as compared to the methods of the prior art. There is a further need for a method of sequencing organic biomolecules that is operable on a biomolecule having any given strand length independent of the size of probe library that is used in the sequencing process.

### BRIEF SUMMARY OF THE INVENTION

In this regard, the present invention provides for sequencing biomolecules such as for example nucleic acids. The method of the present invention uses a nanopore in a manner that allows the detection of the positions (relative and/or absolute) of nucleic acid probes that are hybridized onto a single-stranded nucleic acid molecule whose sequence is of interest (the strand of interest). In accordance with the method of the present invention, as the strand of interest and hybridized probes translocate through the nanopore, the fluctuations in current measured across the nanopore will vary as a function of time. These fluctuations in current are then used to determine the attachment positions of the probes along the strand of interest. This probe position data is then fed into a computer algorithm that returns the sequence of the strand of interest.

In one embodiment of the method of the present invention, the strand of interest is hybridized with the entire library of probes of a given length. For example, the strand of interest can be hybridized with the entire universe of 4096 possible six-mers. The hybridization can be done sequentially (i.e. one probe after another) or in parallel (i.e. a plurality of strands of interest are each separately hybridized simultaneously with each of the possible probes.) Alternatively, the probes can be separated from each other in both space and time. Additionally, more than one probe type may be hybridized to the same strand of interest at the same time.

In another embodiment of the invention, the method is used to sequence very long segments of nucleic acids. An entire genome, for example, is allowed to shear randomly and then each piece of the strand is hybridized and translocated through the nanopore as described above. If it is not known which segment of a genome is being looked at any particular point in time, this can be determined by comparing the pattern of hybridized probes to that which would bind to a reference sequence thereby allowing the location of each fragment to be determined at a later time. This embodiment allows for sequencing of long stretches of nucleic acids without the need for extensive sample preparation. Alternatively, probes of a length different from those used to sequence are first hybridized to the strand of interest in order to mark various locations in the genome. Similarly, proteins known to bind at specific locations along the strand of interest can be used as reference points. It should also be noted that the probe binding pattern can be used to determine the orientation in which the strand of interest translocates through the nanopore (i.e. 5′ to 3′ or 3′ to 5′) by comparing the binding pattern to the reference sequencing in both directions (5′ to 3′ and 3′ to 5′). Alternatively, orientation can be determined by use of a marker that has some directional information associated with it can be attached to the probe (i.e. it gives an asymmetrical signal).

In another embodiment of the invention, probes are separated by (GC) content and other determinants of probe binding strength, in order to allow for optimization of reaction conditions. By separating the probes based on relative properties, multiple probes can be incorporated into a single hybridization reaction. Further, the probes can be grouped based on their related prime reaction environment preferences.

In still another embodiment of the invention, the probes are attached to tags, making the current fluctuations more noticeable as the hybridized probes translocate through the nanopore. In addition, different tags can be used to help distinguish among the different probes. These tags may be proteins or other molecules.

In yet another embodiment of the invention, rolling circle amplification is used to make many copies of the strand of interest or a particular portion of nucleic acid. This gives more data, strengthening the statistical analysis.

In yet another embodiment of the invention, pools of probes are simultaneously hybridized to the strand of interest. A pool of probes is a group of probes of different composition, each of which is likely present in many copies. The composition of the probes would likely be chosen so as not to cause competitive binding to the strand of interest.

Therefore, it is an object of the present invention to provide a method of sequencing a biomolecule using a nanopore device. It is a further object of the present invention to provide a method of sequencing a biomolecule that eliminates the need for time consuming and costly preparation of the biomolecule prior to the sequencing operation. It is still a further object of the present invention to provide a method of sequencing a biomolecule that allows long strands of biomolecules to be sequenced using a nanopore device in a manner that also provides directional information related to the molecule itself.

These together with other objects of the invention, along with various features of novelty that characterize the invention, are pointed out with particularity in the claims annexed hereto and forming a part of this disclosure. For a better understanding of the invention, its operating advantages and the specific objects attained by its uses, reference should be had to the accompanying drawings and descriptive matter in which there is illustrated a preferred embodiment of the invention.

### BRIEF DESCRIPTION OF THE DRAWINGS

In the drawings which illustrate the best mode presently contemplated for carrying out the present invention:

FIG. 1 is a schematic depiction of a DNA molecule;

FIG. 2 is a schematic depiction of an RNA molecule;

FIG. 3 is a schematic depiction of a hybridizing oligonucleotides (A.K.A. probe);

FIG. 4 is a schematic depiction of a single strand DNA molecule hybridized with a probe;

FIG. 5 is a schematic depiction of an apparatus employed in the method of the present invention;

FIG. 6 is a close up view as a hybridized biomolecule translocated through the nanopore of the apparatus in FIG. 5;

FIG. 7 depicts the results from a repetitive application of the method of the present invention using different probes; and

FIG. 8 shows a strand having a bead attached thereto translocating using a magnetic buffer.

### DETAILED DESCRIPTION OF THE INVENTION

As stated above, the present invention is directed to a method of sequencing and mapping strands of organic biomolecules. In the context of the present invention the term biomolecule is intended to include any known form of biomolecule including but not limited to for example DNA, RNA (in any form) and proteins. In basic terms, DNA is the fundamental molecule containing all of the genomic information required in living processes. RNA molecules are formed as complementary copies of DNA strands in a process called transcription. Proteins are then formed from amino acids based on the RNA patterns in a process called translation. The common relation that can be found in each of these molecules is that they are all constructed using a small group of building blocks or bases that are strung together in various sequences based on the end purpose that the resulting biomolecule will ultimately serve.

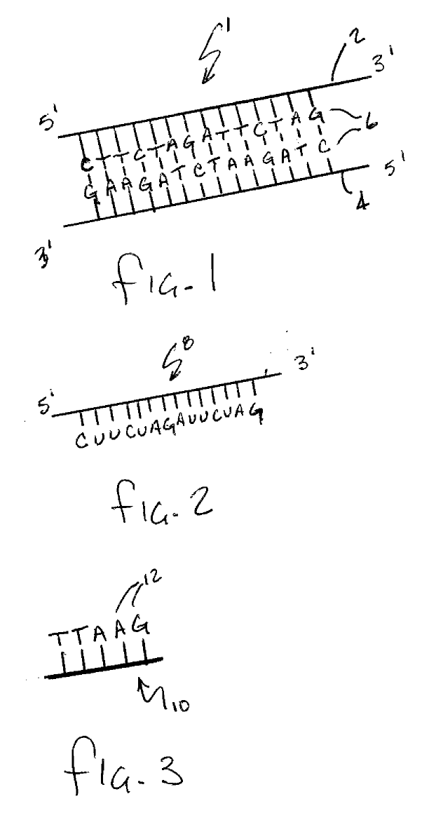

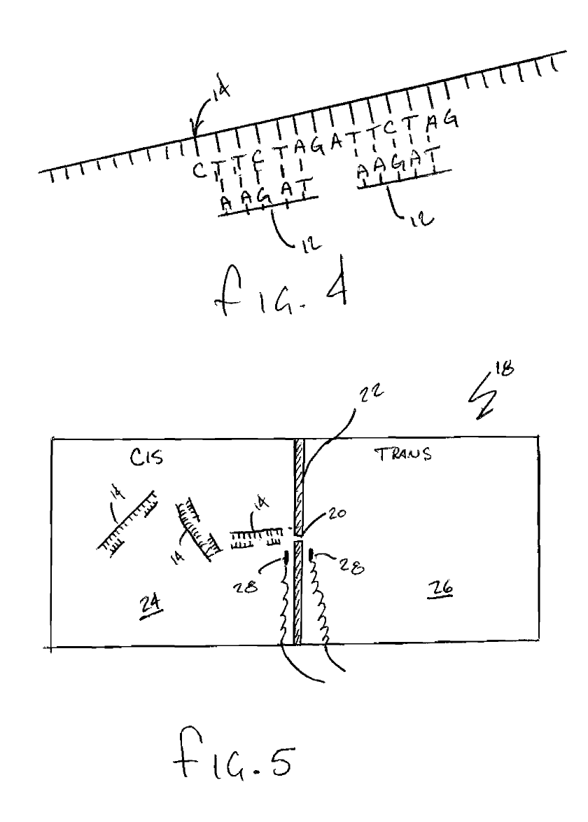

Turning to FIG. 1, a DNA molecule 1 is schematically depicted and can be seen to be structured in two strands 2, 4 positioned in anti-parallel relation to one another. Each of the two opposing strands 2, 4 is sequentially formed from repeating groups of nucleotides 6 where each nucleotide 6 consists of a phosphate group, 2-deoxyribose sugar and one of four nitrogen-containing bases. The nitrogen-containing bases include cytosine (C), adenine (A), guanine (G) and thymine (T). DNA strands 2 are read in a particular direction, from the top (called the 5′ or “five prime” end) to the bottom (called the 3′ or “three prime” end). Similarly, RNA molecules 8, as schematically depicted in FIG. 2 are polynucleotide chains, which differ from those of DNA 1 by having ribose sugar instead of deoxyribose and uracil bases (U) instead of thymine bases (T).

Traditionally, in determining the particular arrangement of the bases 6 in these organic molecules and thereby the sequence of the molecule, a process called hybridization is utilized. The hybridization process is the coming together, or binding, of two genetic sequences with one another. This process is a predictable process because the bases 6 in the molecules do not share an equal affinity for one another. T (or U) bases favor binding with A bases while C bases favor binding with G bases. This binding occurs because of the hydrogen bonds that exist between the opposing base pairs. For example, between an A base and a T (or U) base, there are two hydrogen bonds, while between a C base and a G base, there are three hydrogen bonds.

The principal tool that is used then to determine and identify the sequence of these bases 6 in the molecule of interest is a hybridizing oligonucleotide commonly called a probe 10. Turning to FIG. 3, it can be generally seen that a probe 10 is a known DNA sequence of a short length having a known composition. Probes 10 may be of any length dependent on the number of bases 12 that they include. For example a probe 10 that includes six bases 12 is referred to as a six-mer wherein each of the six bases 12 in the probe 10 may be any one of the known four natural base types A, T(U), C or G and alternately may include non-natural bases. In this regard the total number of probes 10 in a library is dependent on the number of bases 12 contained within each probe 10 and is determined by the formula 4ⁿ(four raised to the n power) where n is equal to the total number of bases 12 in each probe 10. Accordingly, the general expression for the size of the probe library is expressed as 4ⁿ n-mer probes 10. For the purpose of illustration, in the context of a six-mer probe the total number of possible unique, identifiable probe combinations includes 4⁶ (four raised to the sixth power) or 4096 unique six-mer probes 10. It should be further noted that the inclusion of non-natural bases allows the creation of probes that have spaces or wildcards therein in a manner that expands the versatility of the library while reducing the number of probes required to reach the final sequence result.

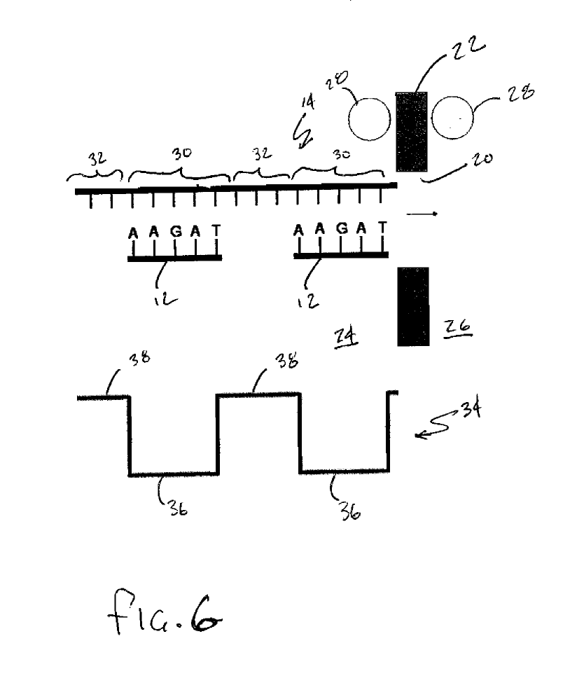

In this context, the process of hybridization using probes 12 as depicted in FIG. 4 first requires that the biomolecule strand be prepared in a process referred to as denaturing. Denaturing is a process, which is accomplished usually through the application of heat or chemicals, wherein the hydrogen bonds between adjacent portions of the biomolecule are broken. For example, the bonds between the two halves of the original double-stranded DNA are broken, leaving a single strand of DNA whose bases are available for hydrogen bonding. After the biomolecule 14 has been denatured, a single-stranded probe 12 is introduced to the biomolecule 14 to locate portions of the biomolecule 14 that have a base sequence that is similar to the sequence that is found in the probe 12. In order to hybridize the biomolecule 14 with the probe 12, the denatured biomolecule 14 and a plurality of the probes 12 having a known sequence are both introduced to a solution. The solution is preferably an ionic solution and more preferably is a salt containing solution. The mixture is agitated to encourage the probes 12 to bind to the biomolecule 14 strand along portions thereof that have a matched complementary sequence. It should be appreciated by one skilled in the art that the hybridization of the biomolecule 14 using the probe 12 may be accomplished before the biomolecule 14 is introduced into a nanopore sequencing apparatus or after the denatured biomolecule 14 has been placed into the cis chamber of the apparatus described below. In this case, after the denatured biomolecule has been added to the cis chamber, a drop of buffer solution containing probes 12 with a known sequence are also added to the cis chamber and allowed to hybridize with the biomolecule 14 before the hybridized biomolecule is translocated.

Once the biomolecule strand 14 and probes 12 have been hybridized, the strand 14 is introduced to one of the chambers of a nanopore sequencing arrangement 18. It should also be appreciated to one skilled in the art that while the hybridization may be accomplished before placing the biomolecule strand 14 into the chamber, it is also possible that the hybridization may be carried out in one of these chambers as well.

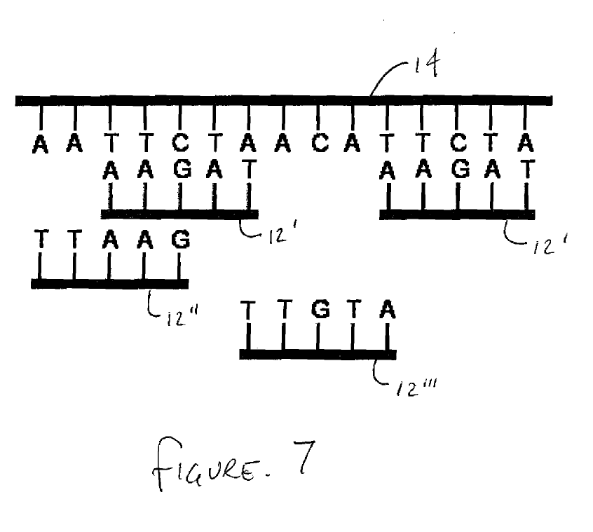

The nanopore sequencing arrangement 18 is graphically depicted at FIG. 5. For the purpose of illustration, relatively short biomolecule strands 14 with only two probes 12 are depicted. It should be appreciated by one skilled in the art that the intent of this depiction is that long strand biomolecule 14 will be translocated through the nanopore 20 to determine the location of the probes 12 attached thereto. The sequencing arrangement 18 includes a nanopore 20 formed in a thin wall or membrane 22. More preferably the nanopore 20 is formed in a solid-state material. Further, it is preferable that the nanopore 20 have a diameter that allows the passage of double stranded DNA and is between approximately 1 nm and 100 nm. More preferably the nanopore has a diameter that is between 2.3 nm and 100 nm. Even more preferably the nanopore has a diameter that is between 2.3 nm and 50 nm. The nanopore 20 is positioned between two fluid chambers, a cis chamber 24 and a trans chamber 26, each of which is filled with a fluid. The cis chamber 24 and the trans chamber 26 are in fluid communication with one another via the nanopore 20 located in the membrane 22. A voltage is applied across the nanopore 20. This potential difference between the chambers 24, 26 on opposing sides of the nanopore 20 results in a measurable ionic current flow across the nanopore 20. Electrodes 28 are installed into each of the cis 24 and trans 26 chambers to measure the difference in electrical potential and flow of ion current across the nanopore 20. The electrode in the cis chamber is a cathode, and the electrode in the trans chamber is an anode.

The hybridized biomolecule strand 14 with the probes 12 attached thereto is then introduced into the cis chamber in which the cathode is located. The biomolecule 14 is then driven or translocated through the nanopore 20 as a result of the applied voltage. As the molecule 14 passes through the nanopore 20, the monitored current varies by a detectable and measurable amount. The electrodes 28 detect and record this variation in current as a function of time. Turning to FIG. 6, it can be seen that these variations in current are the result of the relative diameter of the molecule 14 that is passing through the nanopore 20 at any given time. For example, the portions 30 of the biomolecule 14 that have probes 12 bound thereto are twice the diameter as compared to the portions 32 of the biomolecule 14 that have not been hybridized and therefore lack probes 12. This relative increase in thickness of the biomolecule 14 passing through the nanopore 20 causes a temporary interruption or decrease in the current flow therethrough resulting in a measurable current variation as is depicted in the waveform 34 at the bottom of the figure. As can be seen, as the portions 30 of the molecule 14 that include probes 12 pass through the nanopore 20, the current is partially interrupted forming a relative trough 36 in the recorded current across the entire duration while the bound portion 30 of the molecule passes. Similarly, as the unhybridized portions 32 of the biomolecule 14 pass, the current remains relatively high forming a peak 38 in the measured current. The electrodes 28 installed in the cis 24 and trans 26 chambers detect and record these variations in the monitored current. Further, the measurements of the current variations are measured and recorded as a function of time. As a result, the periodic interruptions or variations in current indicate where, as a function of relative or absolute position, along the biomolecule 14 the known probe 12 sequence has attached.

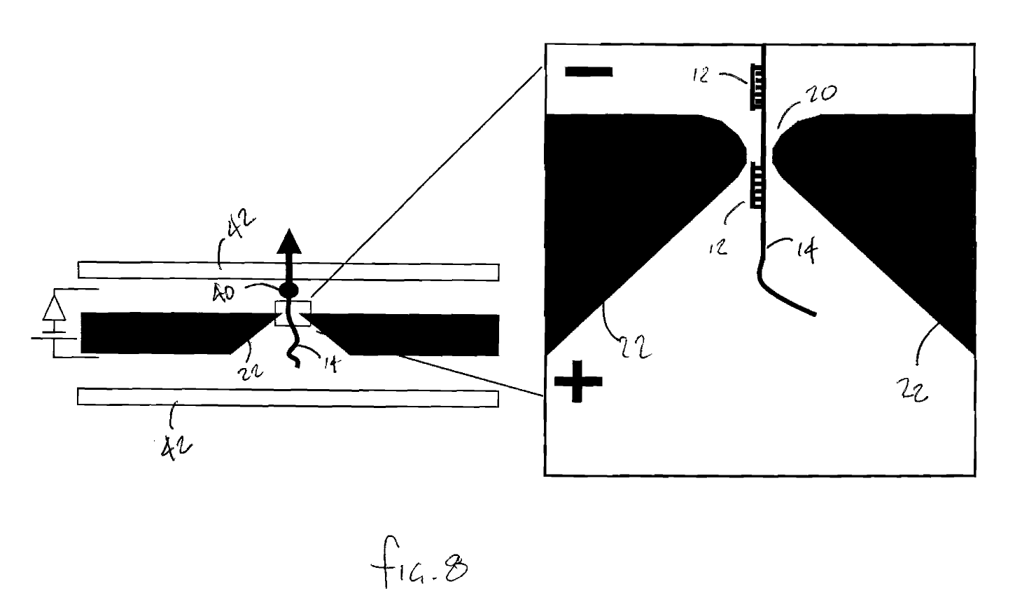

The measurements obtained and recorded as well as the time scale are input into a computer algorithm that maps the binding locations of the known probe 12 sequences along the length of the biomolecule 14. Once the probe 12 locations are known, since the probe 12 length and composition is known, the sequence of the biomolecule 14 along the portions 30 to which the probes 12 were attached can be determined. This process can then be repeated using a different known probe 12. Further, the process can be repeated until every probe 12 within the library on n-mer probes has been hybridized with the biomolecule 14 strand of interest. It can best be seen in FIG. 7 that by repeating the process with different known probes 12′, 12″ and 12′″, the gaps in the portions of the biomolecule 14 are gradually filled in with each subsequent hybridization and sequencing step until eventually the entire sequence of the biomolecule 14 of interest is known.

It should be appreciated by one skilled in the art that each subsequent hybridization and sequencing of the biomolecule 14 via the method of the present invention could be accomplished in a variety of fashions. For example, a plurality of nanopore assemblies, each sequencing copies of the same biomolecule of interest using different known probes may be utilized simultaneously in a parallel fashion. Similarly, the same biomolecule may be repetitively hybridized and sequenced by passing it through a series of interconnected chambers. Finally, any combination of the above two processes could also be employed.

It should also be appreciated that the detection of variations in electrical potential between the cis and trans chambers as the hybridized biomolecule 12 of interest passes through the nanopore 20 may be accomplished in many different ways. For example, the variation in current flow as described above may be measured and recorded. Optionally, the change in capacitance as measured on the nanopore membrane itself may be detected and recorded as the biomolecule passes through the nanopore. Finally, the quantum phenomenon known as electron tunneling, whereby electrons travel in a perpendicular fashion relative to the path of travel taken by the biomolecule. In essence, as the biomolecule 14 passes through the nanopore 20, the locations where the probes 12 are attached thereto bridge the nanopore 20 thereby allowing electrons to propagate across the nanopore in a measurable event . As the electrons propagate across the nanopore the event is be measured and recorded to determine the relative locations to which probes have been bound. The particular method by which the electrical variations are measured is not important, only that fluctuation in electrical properties is measured as they are impacted by the passing of the biomolecule through the nanopore.

It is also important to note that the way in which the electrical potential varies, as a function of time, is dependent on whether a single stranded (un-hybridized) or double stranded (hybridized) region of the biomolecule is passing through the nanopore 20 may be complicated. In the simplest scenario, the double stranded region 30 will suppress the current as compared to the single stranded region 32, which will suppress the current as compared to when no biomolecule 14 is translocating. However, for small nanopore 20 dimensions or high salt concentrations, it is possible that the current may in fact be augmented with the translocation of double-stranded portions 30. In this case, the points of increased current would be used as an indicator as to where the probes 12 are positioned along the biomolecule 14.

The recorded changes in electrical potential across the nanopore 20 as a factor of time are then processed using a computer and compiled using the sequences of the known probes 12 to reconstruct the entire sequence of the biomolecule 14 strand of interest.

The method of the present invention represents a substantial improvement over traditional sequencing-by-hybridization (SBH) methods. The SBH process is extremely inefficient for long strands of DNA of interest. In contrast, the method of the present invention provides both hybridization as well as the relative position of the probe along the biomolecule strand. Due to the addition of the positional information, as is provided via the method of the present invention, a probe library of finite size can be utilized to sequence a strand of interest of arbitrary length. The additional positional information also solves the repeat problem in which repeats of probe binding sites on a long DNA prevent successful reconstruction of the DNA sequence from the sequences of the binding probes. Finally, the addition of positional information as provided by the method of the present invention means that the computational problem of reconstructing the sequence is no longer NP-complete, a mathematical term indicating extreme difficulty, as was the case in traditional SBH processes. It should also be noted that perhaps the most basic improvement of this method as compared to SBH is that it that it gives the number of copies of a given probe that hybridize to the strand of interest.

It should be noted that there is inherent error in resolving the exact probe 12 locations along the strand 14. For example, the resolution error may by on the order of +/− hundreds of bases A great deal of this resolution error can be estimated and incorporated into the algorithm thereby providing a positional binding range of the probes 12 along the strand 14 at the data processing level. While the illustrations contemplate measuring the locations of the bound probes 12 exactly (i.e. to single-base resolution) it should be noted that this it is not necessary to know the locations exactly in order for the algorithm to return an exact and correct sequence. As long as the error can be estimated, it can be taken into account in the algorithm. (By way of comparison, traditional SBH effectively has infinite error in the measurement of the probe locations. ) In addition to the introduction of error correction, in order to improve the quality of the signal, it may be necessary to slow down the speed at which the hybridized biomolecule 14 strand of interest translocates through the nanopore 20. Control over the translocation speed can be achieved in a variety of ways. One such way to control translocation speed is through the use of a viscous fluid solution through which the hybridized biomolecule 14 will have to travel. Another way is the use of optical or magnetic tweezers. In this case, the hybridized biomolecule 14 is attached to a bead 40 and optical or magnetic tweezers 42 are used to drag on the bead 40 to slow down the translocation (see FIG. 8). Yet another method is to use a low-temperature setup, which has the added benefit of reducing signal noise.

In another embodiment of the invention, the method is used to sequence very long segments of nucleic acids. An entire genome, for example, is allowed to shear randomly and then each piece of the strand 14 is hybridized and translocated through the nanopore 20 as described above. While it is not known which segment of a genome is being examinedat any particular point in time, this can be determined by comparing the pattern of hybridized probes 12 to that which would bind to a reference sequence thereby allowing the relative location within the genome of each fragment to be determined at a later time. This embodiment allows for sequencing of long stretches of nucleic acids without the need for extensive sample preparation. Alternatively, probes 12 of a length different from those used to sequence are first hybridized to the strand of interest in order to mark various locations in the genome. Similarly, proteins known to bind at specific locations along the strand of interest can be used as reference points. Such features provide known reference marks at predictable points within the strand to assist in reassembling the sequence in final processing of the sequence information. This also facilitates a determination of the orientation in which the strand of interest translocates through the nanopore (i.e. 5′ to 3′ or 3′ to 5′) by comparing in both directions to the locations of probes in a reference sequence or by the addition of a marker that has some directional information associated with it (i.e. it gives an asymmetrical signal).

In another embodiment of the invention, probes are separated by (GC) content and other determinants of probe binding strength as was described above, in order to allow for optimization of reaction conditions.

In still another embodiment of the invention, the probes 12 are attached to tags.

Such tags may take the form of proteins of other molecules that are attached to the back of each of the probes 12 used in the hybridization. The tags result in an even greater increase the diameter of hybridized biomolecule at the points of probe attachment thereby making the current fluctuations more noticeable as the hybridized probes translocate through the nanopore. In addition, different tags can be used to help distinguish among the different probes.

In yet another embodiment of the invention, rolling circle amplification is used to make many copies of the strand of interest or a particular portion of nucleic acid. This gives more data, strengthening the statistical analysis

It is also possible that when sequencing long lengths of single-stranded strands of interest or strands of RNA, it may be difficult to prevent the molecule from self-hybridizing, i.e. folding back and hybridizing along their own lengths. This can be prevented by placing the hybridized biomolecule into a nano-channel that is coupled to a nanopore such that the nano-channel holds the molecule in a relatively straight position until it passes through the nanopore. Alternatively, or in addition, single-stranded binding proteins can be used to keep the molecule single-stranded.

It can therefore be seen that the present invention provides a novel method for determining the sequence of a biomolecule strand of interest whereby long strands can be sequenced at a relatively high rate of speed and at a lower cost as compared to the prior art. Further, the present invention can be modified to sequence biomolecule of any length and facilitates the reintegration of the various severed portions of the strand in a manner that was heretofore unknown. For these reasons, the method of the present invention is believed to represent a significant advancement in the art, which has substantial commercial merit.

While there is shown and described herein certain specific structures embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed:

1. A method for determining the sequence of a biomolecule strand of interest, comprising the steps of:
providing a sequencing apparatus having a first fluid chamber, a second fluid chamber, a membrane positioned between said first and second chambers and a nanopore extending through said membrane such that said first and second chambers are in fluid communication via said nanopore;
providing a single-stranded biomolecule;
providing a first plurality of matching probes having a known sequence;
hybridizing said first plurality of probes with said single-stranded biomolecule such that said first plurality of probes attach to portions of said single-stranded biomolecule to produce a partially hybridized biomolecule;
introducing said partially hybridized biomolecule into said first chamber;
translocating said partially hybridized biomolecule from said first chamber through said nanopore and into said second chamber;
monitoring changes in current across said nanopore as said partially hybridized biomolecule is translocated therethrough, said changes in electrical potential corresponding to locations along said partially hybridized biomolecule containing one of said first plurality of probes; and
recording said changes in electrical potential as a function of time.

2. The method of claim 1, wherein said method is repeated using a second plurality of matching probes having a known sequence different than said known sequence of said first plurality of probes.

3. The method of claim 1, wherein said probes are hybridizing oligonucleotides having n number of bases therein.

4. The method of claim 3, wherein said method is repeated sequentially by replacing said first plurality of probes with a subsequent plurality of each of the different unique probes within the entire library of 4ⁿ n-mer probes.

5. The method of claim 4, wherein said sequential repetition of said method is conducted in a linear series of reactions.

6. The method of claim 4, wherein said sequential repetition of said method is conducted in a parallel series of reactions

7. The method of claim 1, wherein said recorded changes in electrical potential are processed using a computer algorithm to reconstruct the sequence of the biomolecule strand.

8. The method of claim 1, wherein said translocation is slowed down by introducing a viscous fluid into said first and second chambers.

9. The method of claim 1, wherein said translocation is slowed down by implementing a low temperature setup.

10. The method of claim 1, wherein a bead is attached to said hybridized strand and said translocation is slowed down through the use of optical tweezers.

11. The method of claim 1, wherein said step of hybridization of said biomolecule further comprises the steps of:
introducing said biomolecule to said first fluid chamber;
introducing a drop of a buffer solution containing said first plurality of probes into said first fluid chamber; and
allowing said probes to hybridize with said biomolecule within said first fluid chamber.

12. The method of claim 1 wherein said first fluid chamber is a cis chamber having a cathode and said second fluid chamber is a trans chamber having an anode.

13. The method of claim 1 wherein said membrane is a solid-state membrane having a nanopore formed therein.

14. The method of claim 1, wherein said nanopore has a diameter of between approximately 1 nm and 100 nm.

15. The method of claim 1, wherein said biomolecule strand of interest is selected from the group consisting of DNA, RNA and proteins.

16. The method of claim 1, said sequencing apparatus further including electrodes in said first and second fluid chambers, said electrodes configured to measure changes in electrical potential across said nanopore.

17. The method of claim 1, wherein the step of monitoring changes in electrical potential comprises monitoring changes in current across said nanopore.

18. The method of claim 1, wherein the step of monitoring changes in electrical potential comprises monitoring changes in capacitance across said nanopore.

19. The method of claim 1, wherein the step of monitoring changes in electrical potential comprises monitoring electron tunneling across said nanopore.
